(19) United States
(12) Patent Application Publication
(10) Pub. No.: US 2011/0236506 A1
(43) Pub. Date: Sep. 29, 2011

(54) PHARMACEUTICAL ASSOCIATION CONTAINING LIPOIC ACID AND HYDROXYCITRIC ACID AS ACTIVE INGREDIENTS

(75) Inventors: Laurent SCHWARTZ, Paris (FR); Adeline GUAIS-VERGNE, Draveil (FR)

(73) Assignees: Laurent SCHWARTZ, Paris (FR); BIOREBUS, Paris (FR)

(21) Appl. No.: 13/099,897

(22) Filed: May 3, 2011

Related U.S. Application Data
(63) Continuation of application No. PCT/FR2009/052110, filed on Nov. 2, 2009.

(30) Foreign Application Priority Data
Nov. 3, 2008 (FR) 0857448

(51) Int. Cl. A61K 33/24; A61K 31/385; A61K 31/685; A61K 31/4985; A61K 31/7056; A61P 35/00

(52) U.S. Cl. 424/649; 514/440; 514/77; 514/249; 514/52

(57) ABSTRACT

Pharmaceutical combination containing lipoic acid and hydroxycitric acid as active ingredients. The present invention relates to a novel pharmaceutical combination and to the use thereof for producing a medicament having an antitumor activity. According to the invention, this combination comprises, as active ingredients: lipoic acid or one of the pharmaceutically acceptable salts thereof; and hydroxycitric acid or one of the pharmaceutically acceptable salts thereof. Said active ingredients being formulated together or separately for a conjugated, simultaneous or separate use.

The subject of the present invention is a novel pharmaceutical combination comprising, as active ingredients, lipoic acid or one of the pharmaceutically acceptable salts thereof, and hydroxycitric acid or one of the pharmaceutically acceptable salts thereof.

This pharmaceutical combination, which can be in the form of single dosage units or in kit form, has a particularly high antitumor activity.

Lipoic acid is a cofactor for several enzyme complexes.

Lipoic acid is also a powerful antioxidant. It assists in protecting cells against damage by free radicals.

This product is known as an active ingredient of medicaments. It is in particular recommended for the treatment of diabetes-related neuropathies, of mitochondrial myopathies and of multiple sclerosis.

It is perfectly tolerated and its toxicity is very low. By way of example, it can be administered in an amount of between 600 and 1800 mg/d.

The use of lipoic acid or one of the water-soluble salts thereof, alone or in combination with ascorbic acid, has been recommended in the treatment of cancer, in particular by document WO 00/48594 corresponding to U.S. Pat. No. 6,448,287.

Moreover, the use of lipoic acid derivatives or the pharmaceutically acceptable salts thereof has also been recommended in the treatment of neoplastic diseases by document WO 00/24734 corresponding to U.S. Pat. No. 6,951,887.

However, no medicament based on lipoic acid or one of the derivatives thereof or one of the pharmaceutically acceptable salts thereof appears, at this time, to be in the process of development for cancer treatment.

Hydroxycitric acid is a natural product which is found in the natural state in the skins of the fruits of the Malabar tamarind (Garcinia). The calcium salt thereof (calcium hydroxycitrate) is known to inhibit fatty acid biosynthesis.

The use of calcium hydroxycitrate has thus been recommended for weight loss, in combination with a low-fat diet, the recommended dosage being from 500 mg to 1500 mg three times a day before meals.

It is a substance that is perfectly well tolerated in adults and in children.

In parallel, calcium hydroxycitrate is known to increase the oxidation of fatty acids in hepatic cells, thereby allowing the conversion of said fatty acids to glycogen. The glycogen is then stored in the muscles so as to be available in the event of physical exercise. Hydroxycitrate is thus used in many dietetic diets for the treatment of obesity. It in particular has the advantage of not modifying the blood glucose level. U.S. Pat. No. 6,207,714 emphasizes that hydroxycitrate can be used as a hypoglycemic agent for treating individuals suffering from insulin-resistant diabetes.

Moreover, hydroxycitrate is mentioned among the very large number of compounds that can be used in the treatment of cancer cells having a high rate of aerobic glycolysis (document WO 2004/100885).

In this context, it has been discovered, and this constitutes the basis of the present invention, that the combination of lipoic acid or one of the pharmaceutically acceptable salts thereof and of hydroxycitric acid or one of the pharmaceutically acceptable salts thereof has a particularly high antitumor activity resulting from a synergistic effect of the constituent active ingredients thereof.

It has in particular been demonstrated that this novel combination makes it possible to limit tumor growth in an entirely unexpected manner, the volume of said tumors stabilizing over a period of at least 100 days at values substantially equal to the volume of the tumors at the beginning of treatment. This tumor stabilization effect is, surprisingly, greater than that obtained by means of known anticancer medicaments.

Consequently, the combination which is the subject of the present invention is particularly original owing to the potentiating synergy of the actions of each of these active ingredients.

Thus, according to a first aspect, the present application aims to cover a pharmaceutical combination which comprises:
firstly, lipoic acid or one of the pharmaceutically acceptable salts thereof; and
secondly, hydroxycitric acid or one of the pharmaceutically acceptable salts thereof.

In the context of the present description, the term “lipoic acid” is intended to cover the compound which exists in the acid form and also the compound which exists in the reduced form, also known as dihydrolipoic acid and its pharmaceutical acceptable salts.

Moreover, lipoic acid and hydroxycitric acid have, respectively, 1 and 2 asymmetric carbon atoms. They can therefore exist in the form of enantiomers or diastereoisomers. These enantiomers and diastereoisomers, and also mixtures thereof, including racemic mixtures, are part of the invention. Preferably, the R form of lipoic acid and the 2S, 3S form of hydroxycitric acid will be used.

In general, the two active ingredients characterizing the pharmaceutical combination according to the invention can be formulated together (single dosage units) or separately (kit).

Also in general, the two active ingredients, formulated together or separately, can be administered simultaneously or separately with a time interval which can be desirable for optimization of their conjugated action in view of the nature of their respective formulation.

A pharmaceutically acceptable salt of lipoic acid can be a water-soluble salt as described in U.S. Pat. No. 6,448,287.

A pharmaceutically acceptable salt of hydroxycitric acid can be an alkali metal (in particular sodium) or alkaline-earth metal (in particular calcium or magnesium) salt.

The pharmaceutical combinations of the invention comprise the two active ingredients identified above. According to one particular embodiment, they comprise no other active ingredient. Alternatively, the presence of at least one other active ingredient in these novel combinations can be envisioned.

Thus, it has been shown that the efficacy of the pharmaceutical combinations of the invention comprising the two active ingredients identified above can be improved when these two active ingredients are combined with at least one additional active ingredient chosen from the group consisting of cisplatin, capsaicin, choline, miltefosine and vitamin B12.

Multiple pharmaceutical combinations which are preferred in this context are, in particular:
triple combinations consisting of the two active ingredients identified above and an additional active ingredient chosen from cisplatin, capsaicin and methotrexate;
multiple combinations consisting:
   either of the two active ingredients identified above, capsaicin and cisplatin;
   or of the two active ingredients identified above, capsaicin, miltefosine, choline and vitamin B12.

According to a first embodiment of the invention, which is currently preferred, the above-mentioned pharmaceutical combination consists of single dosage units incorporating the active ingredients usually in a pharmaceutically acceptable excipient.

According to a second embodiment, this pharmaceutical combination is in the form of a kit containing:
firstly, lipoic acid or one of the pharmaceutically acceptable salts thereof, usually in a pharmaceutically acceptable excipient; and
secondly, hydroxycitric acid or one of the pharmaceutically acceptable salts thereof, usually in a pharmaceutically acceptable excipient.

Regardless of the embodiment envisioned, the nature and the respective amounts of the various excipients may be readily determined by those skilled in the art according to the final dosage form desired.

Preferably, in the case of single dosage units, the active ingredients will be conditioned in a dosage form suitable for oral administration. However, other routes of administration, for instance intramuscular, intravenous, topical or cutaneous routes, can be envisioned.

Likewise, preferably, in the case where the active ingredients are provided separately, they will be conditioned independently of one another, each in a dosage form suitable for oral administration. However, other routes of administration can be envisioned for each of the two dosage forms, independently.

According to one particular characteristic, a dosage form suitable for the oral route can be chosen from tablets, gelatin capsules, powders, granules, lyophilisates, oral solutes and syrups.

However, tablets constitute the currently preferred dosage form suitable for the oral route. These tablets may be of varied nature, immediate-release, controlled-release or delayed-release, and optionally in effervescent or orodispersible form.

In general, the dosage will be adjusted according to the route of administration and the patient to be treated.

Lipoic acid or one of the pharmaceutically acceptable salts thereof can thus be administered, in one, two or three intakes, in an amount of from 0.1 to 100 mg/kg/d, preferably from 1 to 60 mg/kg/d and more preferably from 5 to 40 mg/kg/d.

Hydroxycitric acid or one of the pharmaceutically acceptable salts thereof can be administered, for its part, in one, two or three intakes, in an amount of from 0.1 to 160 mg/kg/d, preferably from 1 to 100 mg/kg/d and more preferably from 15 to 70 mg/kg/d.

By way of example, in the pharmaceutical combination according to the invention, the lipoic acid or one of the pharmaceutically acceptable salts thereof can be present in an amount of between 20 and 800 mg, preferably between 50 and 700 mg, while the hydroxycitric acid can be present in an amount of between 200 and 2000 mg, preferably between 600 and 1600 mg, with a view to an administration at a rate of two or three times a day (NB: the amounts indicated are calculated to be administered 3 times a day). An illustrative combination of the invention can thus comprise 600 mg of lipoic acid and 1200 mg of hydroxycitric acid in a pharmaceutically acceptable excipient for a composition to be taken three times a day.

The pharmaceutical combinations of the invention can be prepared in the usual manner. This preparation comprises:
either the formulation of the active ingredients together in a pharmaceutically acceptable excipient;
or the separate formulation of each of the active ingredients in pharmaceutically acceptable excipients which may be identical or different.

The formulating techniques which can be used for this purpose are well known to those skilled in the art and comprise essentially physical mixing of the active ingredient(s) with the pharmaceutically acceptable excipient(s).

Given the high antitumor activity of the combination which has just been described, the invention is particularly useful for the production of medicaments intended for the treatment of a cell proliferation disease, chosen from the group comprising breast cancer, ovarian cancer, cervical cancer, prostate cancer, testicular cancer, esophageal cancer, stomach cancer, skin cancer, lung cancer, bone cancer, colon cancer, pancreatic cancer, thyroid cancer, bile duct cancer, cancer of the buccal cavity and of the pharynx (oral region), cancer of the lips, of the tongue, of the mouth, of the pharynx or of the small intestine, colorectal cancer, cancer of the large intestine, rectal cancer, cancer of the brain and of the central nervous system, a glioblastoma, a neuroblastoma, a keratoacanthoma, an epidermoid carcinoma, a large cell carcinoma, an adenocarcinoma, an adenoma, a follicular carcinoma, an undifferentiated carcinoma, a papillary carcinoma, a seminoma, a melanoma, a sarcoma, a bladder carcinoma, a liver carcinoma, a kidney carcinoma, myeloid disorders, lymphoid disorders, Hodgkin's disease, a carcinoma with hairy cells, and leukemia.

The pharmaceutical combinations of the invention can of course be used in a therapeutic treatment as a supplement to other anticancer treatments.

The pharmaceutical combination of the invention may thus be used with one or more other active agents, in which case the combination of the invention and the other agent(s) may be administered as part of the same or separate dosage forms, via the same or different routes of administration, and on the same or different administration schedules according to standard pharmaceutical practice.

The combination of the present disclosure intended for pharmaceutical use may be administered alone or in combination with one or more other drugs (or as any combination thereof), in particular with one or more other anti-cancer agents. The combination of the present invention may also be administered alone or in combination with an another active agent as a formulation in association with one or more pharmaceutically acceptable excipients.

Preferably, the anti-cancer agent is a chemical or biological substance which is clinically shown to treat cancer. More preferably, the anti-cancer agent is selected from the group consisting of actinomycin D, adriamycin, amsacrine, ara-C, 9-(3-D-arabinosyl-2-fluoroadenine, BCNU, bleomycin, camptothecin, carboplatin, 2-chloro-2-deoxyadenosine, CPT-11, cyclophosphamide, docetaxel, doxorubicin, edotecarin, etoposide, fludarabine, 5-fluorouracil (5-FU), gemcitabine, HU-Gemzar, Irinotecan, methotrexate, 6-Mpurine, mytomicin-C, paclitaxel, cis-platin, SN-38, taxol, thiotepa, 6-thioguanine, trimetrexate vinblastine, vincristine, and VP-16.

In a particular embodiment, the anti-cancer agent is a DNA damaging agent. Preferably, the “DNA damaging agent” is a chemical or biological substance that is clinically shown to treat cancer. More preferably, the DNA damaging agent is selected from the group consisting of alkylating agents, antimetabolites, antitumor antibiotics, platinum analogs and other metal analogs such as gallium, gold, ruthenium, arsenic, palladium, cobalt, copper and lanthanum analogs, topoisomerase I inhibitors and topoisomerase II inhibitors.

Preferably, the alkylating agent is selected from the group consisting of apaziquone, altretamine, brostallicin, bendamustine, busulfan, carboquone, carmustine, chlorambucil, chlormethine, cyclophosphamide, estramustine, fotemustine, glufosfamide, ifosfamide, lomustine, mafosfamide, mechlorethamine oxide, mecillinam, melphalan, mitobronitol, mitolactol, nimustine, nitrogen mustard N-oxide, pipobroman, ranimustine, temozolomide, thiotepa, treosulfan, and trofosframide.

Preferably, the antimetabolite is selected from the group consisting of Alimta, Ara-C, 5-azacitidine, capecitabine, carmofur, cladribine, clofarabine, cytarabine, cytosine arabinoside, decitabine, disodium premetrexed, doxifluridine, eflornithine, enocitabine, ethynylcytidine, floxuridine, fludarabine, 5-fluorouracil (5-FU), gemcitabine, hydroxyurea, leucovorin, melphalan, 6-mercaptopurine, methotrexate, mitoxantrone, 6-Mpurine, pentostatin, pelitrexol, raltitrexed, riboside, methotrexate, mercaptopurine, nelarabine, nolatrexed, ocfosfate, tegafur, 6-thioguanine (6-TG), tioguanine, triapine, trimetrexate, vidarabine, vincristine, vinorelbine and UFT.

Preferably, the antitumor antibiotic is selected from the group consisting of aclarubicin, actinomycin D, amrubicin, annamycin, adriamycin, bleomycin, dactinomycin, daunorubicin, doxorubicin, elsamitrucin, epirubicin, galarubicin, idarubicin, mitomycin C, mycophenolic acid, nemorubicin, neocarzinostatin, pentostatin, peplomycin, pirarubicin, rebeccamycin, stimalamer, streptozocin, valrubicin and zinostatin.

Preferably, the platinum analogue is selected from the group consisting of carboplatin (Paraplatin), cisplatin, Eloxatin (oxaliplatin, Sanofi), eptaplatin, lobaplatin, nedaplatin, satraplatin and picoplatin, but other platinum compounds may be potentiated by the rhenium complexes of the invention.

Preferably, the topoisomerase I inhibitor is selected from the group consisting of BN-80915 (Roche), camptothecin, CPT-11, edotecarin, exatecan, irinotecan, orathecin (Supergen), SN-38, and topotecan.

Preferably, the toposimerase II inhibitor is selected from amsacrine, etoposide, etoposide phosphate and epirubicin (Ellence).

In another embodiment, the anti-cancer agent is a mitotic inhibitor. Preferably, the mitotic inhibitor is selected from the group consisting of docetaxel (Taxotere), estramustine, paclitaxel, cabazitaxel, razoxane, taxol, teniposide, vinblastine, vincristine, vindesine, vinorelbine and vinflunine.

In another embodiment, the anti-cancer agent is an anti-angiogenesis agent. Preferably, the anti-angiogenesis agent is selected from EGF inhibitors, EGFR inhibitors, VEGF inhibitors, VEGFR inhibitors, TIE2 inhibitors, IGF1R inhibitors, COX-II (cyclooxygenase II) inhibitors, MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif. Additional VEGF inhibitors include CP-547,632 (Pfizer Inc., NY, USA), axitinib (Pfizer Inc.), ZD-6474 (AstraZeneca), AEE788 (Novartis), AZD-2171, VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof.

Preferred EGRF inhibitors include, but are not limited to Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (WAX), EGFR fusion protein, EGF-vacdne, anti-EGFr immunoliposomes (Hermes Biosciences Inc.) and combinations thereof.

Other anti-angiogenic agent include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

In another embodiment, the anti-cancer agent is a pan kinase inhibitor. Preferred pan kinase inhibitors include Sutent™ (sunitinib), described in U.S. Pat. No. 6,573,293.

In another embodiment, the anti-cancer agent is a poly (ADP-ribos) polymerase (PARP1) inhibitor. Preferred PARP1 inhibitors include iniparib (Sanofi).

In another embodiment, the anti-cancer agent is selected from pan Erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifunctional bispecific antibodies (University of Munich) and mAB AR-209 (Aronex Pharmaceuticals Inc) and mAB 2B-1 (Chiron) and combinations thereof.

In another embodiment, the anti-cancer agent is selected from Genasense (augmerosen, Genta), Panitumumab (Vectibix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

In another embodiment, the anti-cancer agent is selected from CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof.

In another embodiment, the anti-cancer agent is selected from Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

In another embodiment, the anti-cancer agent is selected from CeaVac (CEA), NeuTrexin (trimetresate glucuronate) and combinations thereof. Additional anti-tumor agents may be selected from the following agents, OvaRex (oregovomab), Osidem (IDM-1), and combinations thereof. Additional anti-tumor agents may be selected from the following agents, Advexin (ING 201), Tirazone (tirapazamine), and combinations thereof. Additional anti-tumor agents may be selected from the following agents, RSR13 (efaproxiral), Cotara (131I chTNT 1/b), NBI-3001 (IL-4) and combinations thereof. Additional anti-tumor agents may be selected from the following agents, Canvaxin, GMK vaccine, PEG Interon A, Taxoprexin (DHA/paciltaxel), and combinations thereof.

In another embodiment, the anti-cancer agent is selected from drugs targeting (directly or not) pyruvate kinase (notably M2 isoform) activation, PFKFB3, IDH, Nampt inhibitor, SIRT-1, heat shock protein inhibitor (HSP90 Ganetespib, Synta Pharmaceuticals), Transketolase like-1 (TKTL-1), drugs allowing carbonic anhydrase inhibition, Phosphoenol pyruvate carboxykinase (PEPCK) inhibition, NADH dehydrogenase inhibition, lipotropic factors, phospholipase D inhibition, lactacte dehydrogenase inhibition, phosphoenol pyruvate carboxykinase inhibition, cytochrome P450 isoenzymes inhibition, hexokinase inhibition, AMP-activated protein kinase (AMPK) activation, choline kinase inhibition, phospholipase A2 inhibition, Insulin Growth Factor Binding Protein (IGFBP) activation, Citrate synthase inhibition, ATP sensitive potassium channel blocker, Protein Phosphatase 2A (PP2A) activation.

Preferably, the combination of the invention is used with one or more of an active agent selected from the group consisting of gemcitabine, leucovorin, 5-fluorouracil, oxaliplatin, docetaxel, capecitabin, epirubicin, thalidomide and vinorelbin.

The term “excipient” is used herein to describe any ingredient other than the compound(s) of the invention and includes ingredients such as vehicles, carriers, diluents, preservatives and the like. The choice of excipient(s) will largely depend on factors such as the particular mode of administration, the effect of the excipient(s) on solubility and stability, and the nature of the dosage form.

Preferably, the pharmaceutical combinations of the invention is used in combination with at least one other above-mentioned active agents.

Thus, according to a second aspect, the present application aims to cover the use of a pharmaceutical combination as described above, for producing a medicament having an antitumor activity, intended in particular for the treatment of the above-mentioned diseases.

Finally, the present application aims to cover a method for treating the above-mentioned diseases, comprising the administration of a therapeutically effective amount of a combination as described above, to a patient needing same.

### Definitions:

As used herein, the phrase “pharmaceutically acceptable” indicates that the designated carrier, vehicle, diluent, excipient, salt or prodrug is generally chemically and/or physically compatible with the other ingredients comprising a formulation, and is physiologically compatible with the recipient thereof.

The phrases “therapeutic” and “therapeutically effective amount” as used herein denote an amount of a compound, composition, medicament or pharmaceutical combination that (a) treats or prevents a particular disease, condition or disorder; (b) attenuates, ameliorates or eliminates one or more symptoms of a particular disease, condition or disorder; (c) prevents or delays the onset of one or more symptoms of a particular disease, condition or disorder described herein. It should be understood that the terms “therapeutic” and “therapeutically effective” encompass any one of the aforementioned effects (a)-(c), either alone or in combination with any of the others (a)-(c).

Representative pharmaceutically acceptable salts include, but are not limited to, acetate, aspartate, benzoate, besylate, bicarbonate/carbonate, bisulphate/sulphate, borate, carnsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like. The invention further includes mixtures of salt forms.

Compounds of the combination of the present invention may be administered as prodrugs. The term “prodrug” refers to a compound that is transformed in vivo to yield a compound of Formula I or a pharmaceutically acceptable salt or solvate of the compound. The transformation may occur by various mechanisms, such as via hydrolysis in blood.

A prodrug of a compound of the combination of the invention may be formed in a conventional manner with one or more functional groups in the compound, such as an amino, hydroxyl or carboxyl group. For example, if a compound of the present invention contains a carboxylic acid functional group, a prodrug can comprise: (1) an ester formed by the replacement of a hydrogen of the acid group with a group such as (C₁-C₆)alkyl or (C₆-C₁₀) aryl; (2) an activated ester formed by the replacement of the hydrogen of the acid group with groups such as —(CR₂)COOR′, where CR₂ is a spacer and R can be groups such as H or methyl and R′ can be groups such as (C₁-C₆)alkyl or (C₆-C₁₀)aryl; and/or (3) a carbonate formed by the replacement of the hydrogen of the acid with groups such as CHROCOOR′ where R can be groups such as H or methyl and R′ can be groups such as (C₁-C₆)alkyl or (C₆-C₁₀)aryl.

Discussions regarding prodrugs and their use can be found in, for example, “Prodrugs as Novel Delivery Systems,” T. Higuchi and W. Stella, Vol. 14 of the AS Symposium Series, and Bioreversible Carriers in Drug Design, Pergamon Press, 1987 (ed. E B Roche, American Pharmaceutical Association).

A pharmaceutical combination of the invention, for example, includes forms suitable for oral administration as a tablet, capsule, pill, powder, sustained release formulations, solution, suspension, or for parenteral injection as a sterile solution, suspension or emulsion. Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

In one preferred embodiment, the combination of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations. Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. The compounds of the invention may also be used in fast-dissolving, fast-disintegrating dosage forms such as those described in Expert Opinion in Therapeutic Patents, 11 (6), 981-986 by Liang and Chen (2001).

In another preferred embodiment, the combination of the invention may be administered by parenteral injection. Exemplary parenteral administration forms include sterile solutions, suspensions or emulsions of the compounds of the invention in sterile aqueous media, for example, aqueous propylene glycol or dextrose. In another embodiment, the parenteral administration form is a solution. Such parenteral dosage forms can be suitably buffered, if desired.

Dosage regimens of the compounds and/or pharmaceutical composition/combination of the invention may be adjusted to provide the optimum desired response. For example, a single bolus may be administered, several divided doses may be administered over time or the dose may be proportionally reduced or increased as indicated by the exigencies of the therapeutic situation. The appropriate dosing regimen, the amount of each dose administered and/or the intervals between doses will depend upon the compound of the invention being used, the type of pharmaceutical composition, the characteristics of the subject in need of treatment and the severity of the condition being treated.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It should be noted that variation in the dosage will depend on the compound employed, the mode of administration, the treatment desired and the disorder (severity and type) to be treated or alleviated. The present invention also encompasses sustained release compositions and ‘flash’ formulations, i.e. providing a medication to dissolve in the mouth.

It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

The invention will now be illustrated by the following nonlimiting examples and by the appended figures which show, respectively:

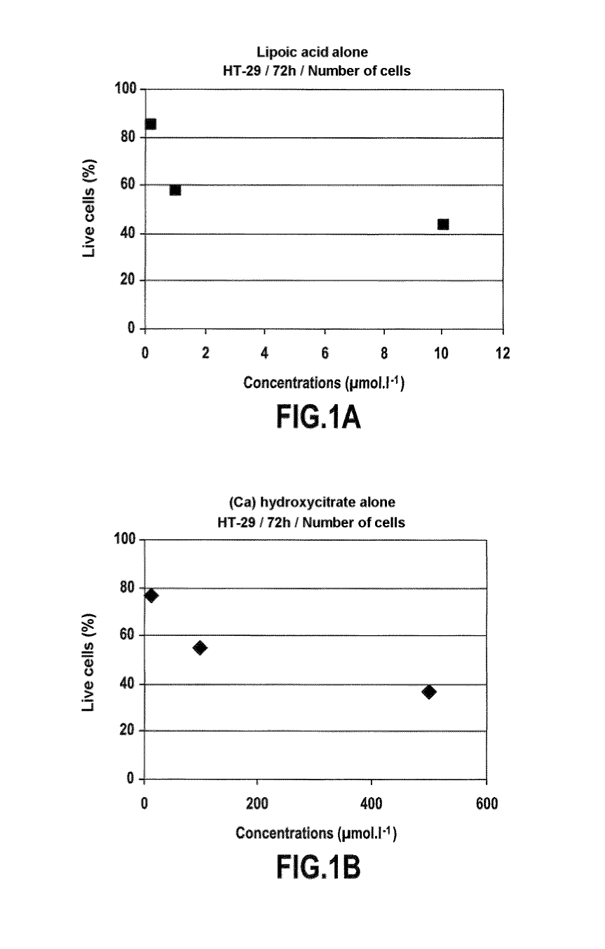

FIGS. 1A and 1B: graphical representation of the results of the cell viability tests using the HT-29 cell line treated with lipoic acid alone (FIG. 1A) or with calcium hydroxycitrate alone (FIG. 1B);

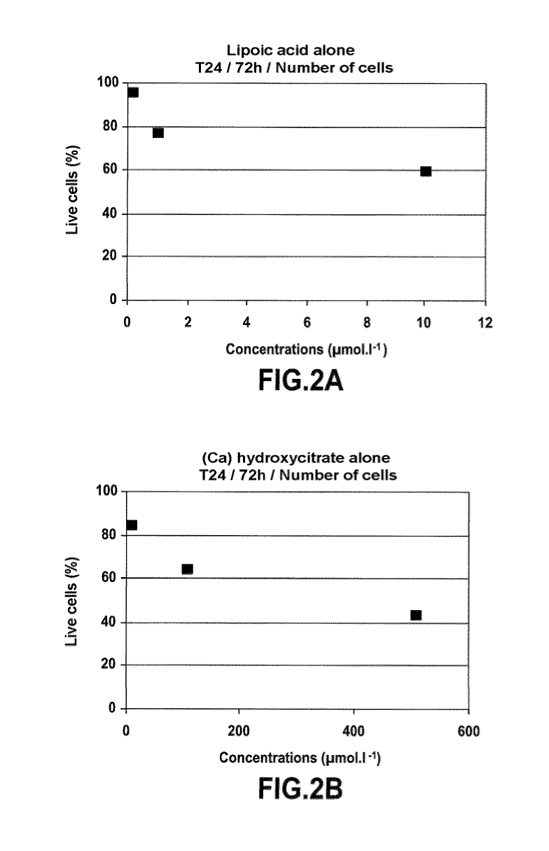

FIGS. 2A and 2B: graphical representation of the results of the cell viability tests using the T-24 cell line treated with lipoic acid alone (FIG. 2A) or with calcium hydroxycitrate alone (FIG. 2B);

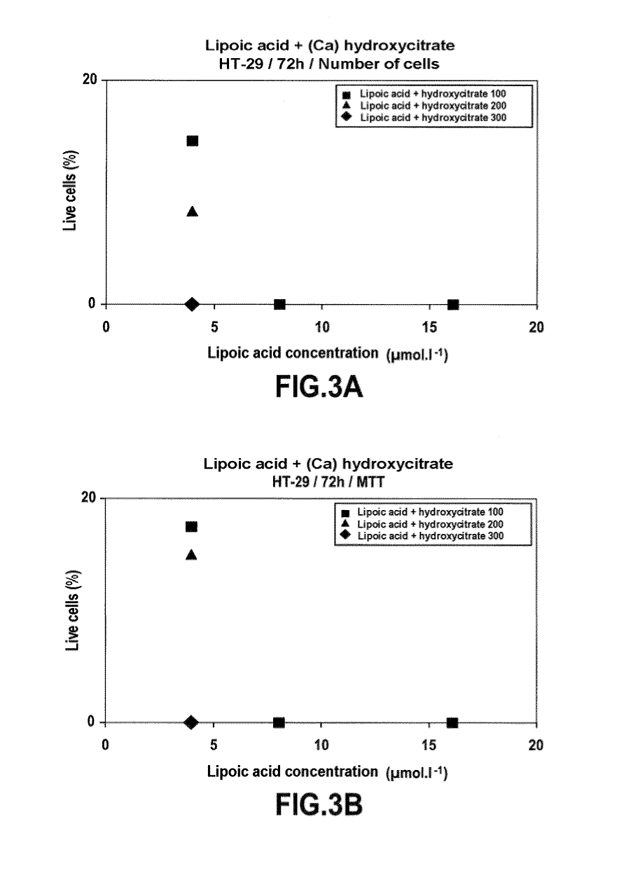

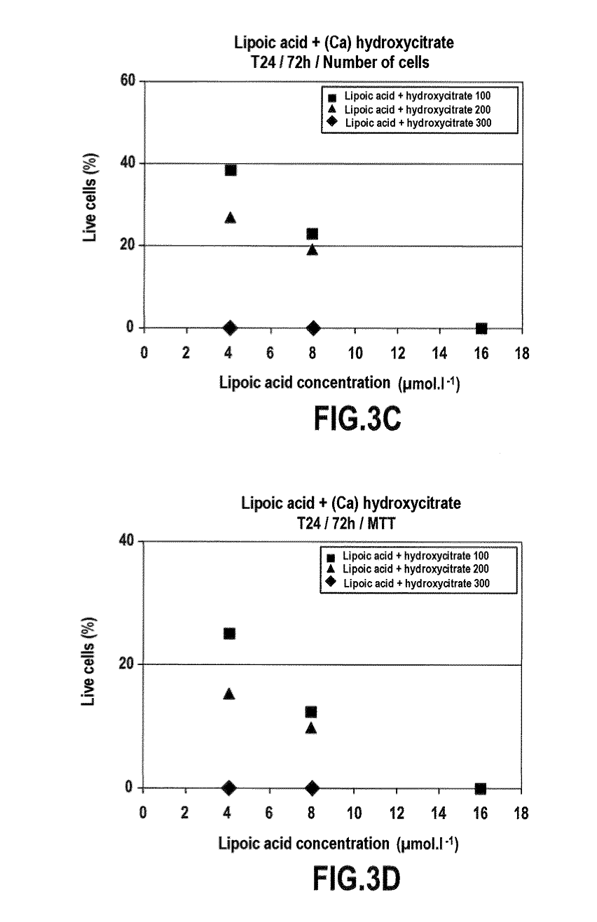

FIGS. 3A to 3D: graphical representation of the results of the cell viability tests using the HT-29 and T-24 cell lines treated with a combination of lipoic acid and calcium hydroxycitrate according to the invention, for three increasing concentrations of calcium hydroxycitrate;

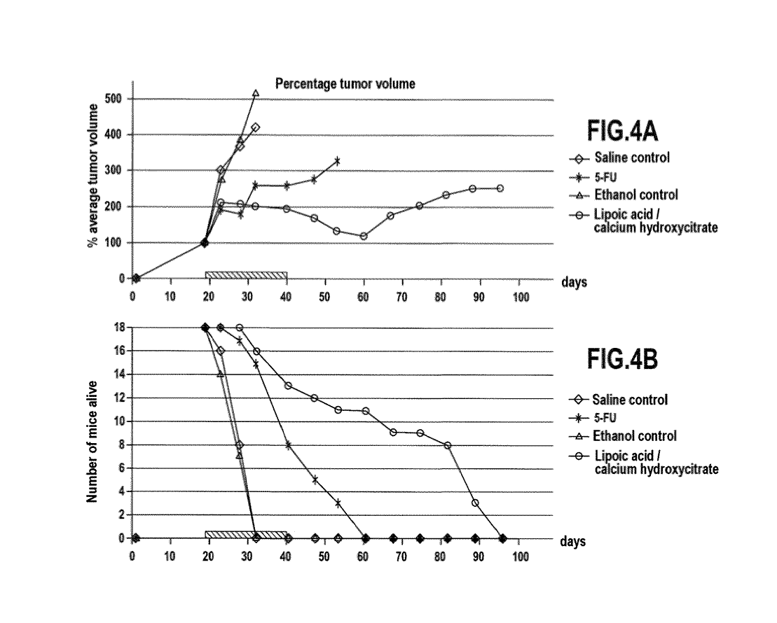

FIG. 4A: graphical representation of the results of treatment of mice with the pharmaceutical combination according to the invention;

FIG. 4B: graphical representation showing the survival of the mice treated in the study, the results of which are shown in FIG. 4A.

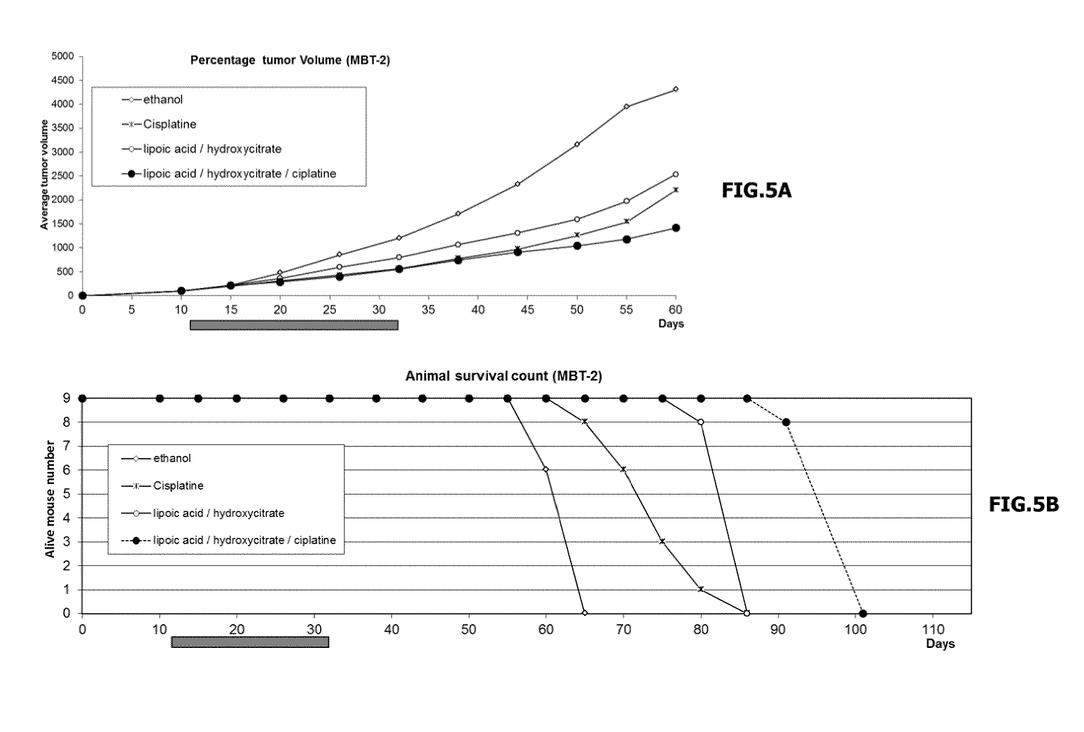

FIG. 5A: graphical representation representing the evolution of the average MTB2 tumor volume during a pharmaceutical treatment of the invention;

FIG. 5B: graphical representation representing the survival rate of the tested mice implanted with MBT2 type tumors during a pharmaceutical treatment of the invention.

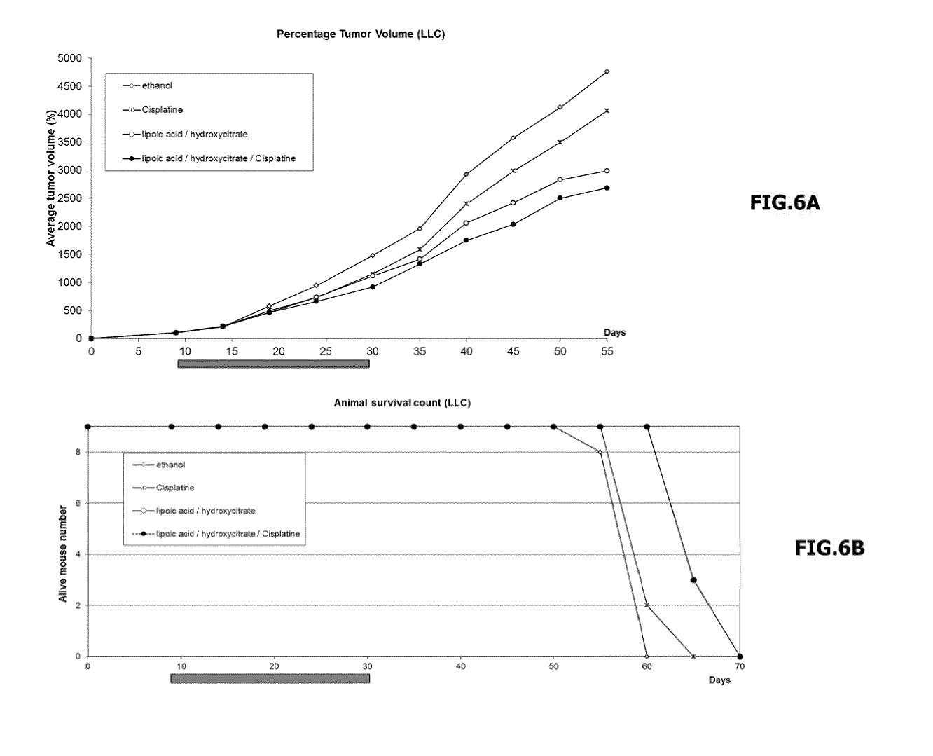

FIG. 6A: graphical representation representing the evolution of the average LLC tumor volume during a pharmaceutical treatment of the invention;

FIG. 6B: graphical representation representing the survival rate of the tested mice implanted with LLC type tumors during a pharmaceutical treatment of the invention.

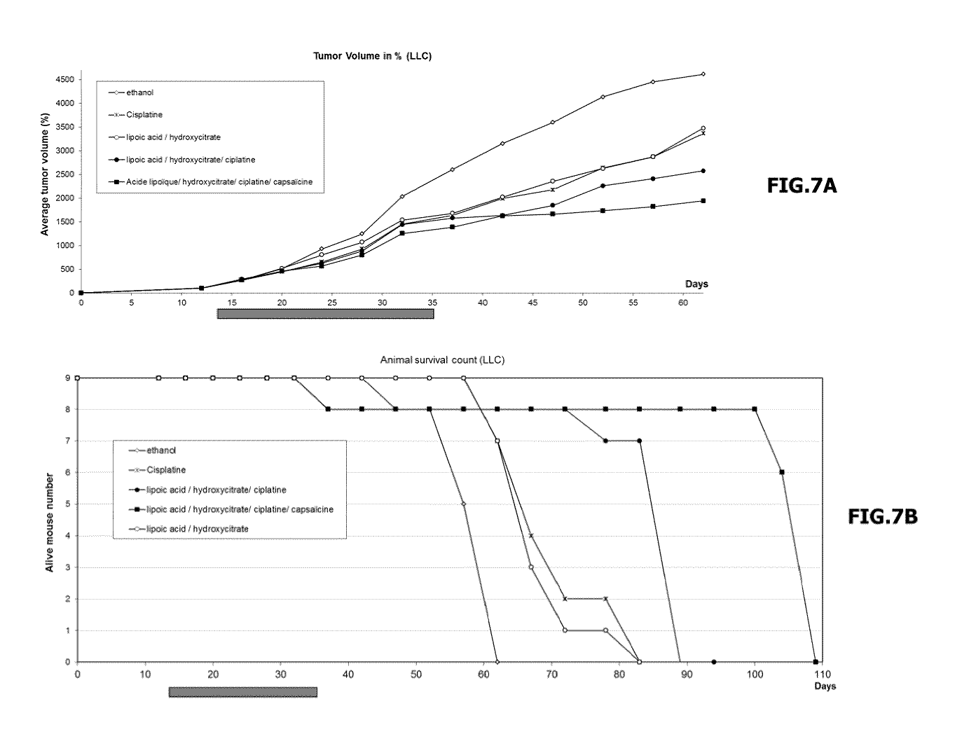

FIG. 7A: graphical representation representing the evolution of the average LLC tumor volume during a pharmaceutical treatment of the invention;

FIG. 7B: graphical representation representing the survival rate of the tested mice implanted with LLC type tumors during a pharmaceutical treatment of the invention.

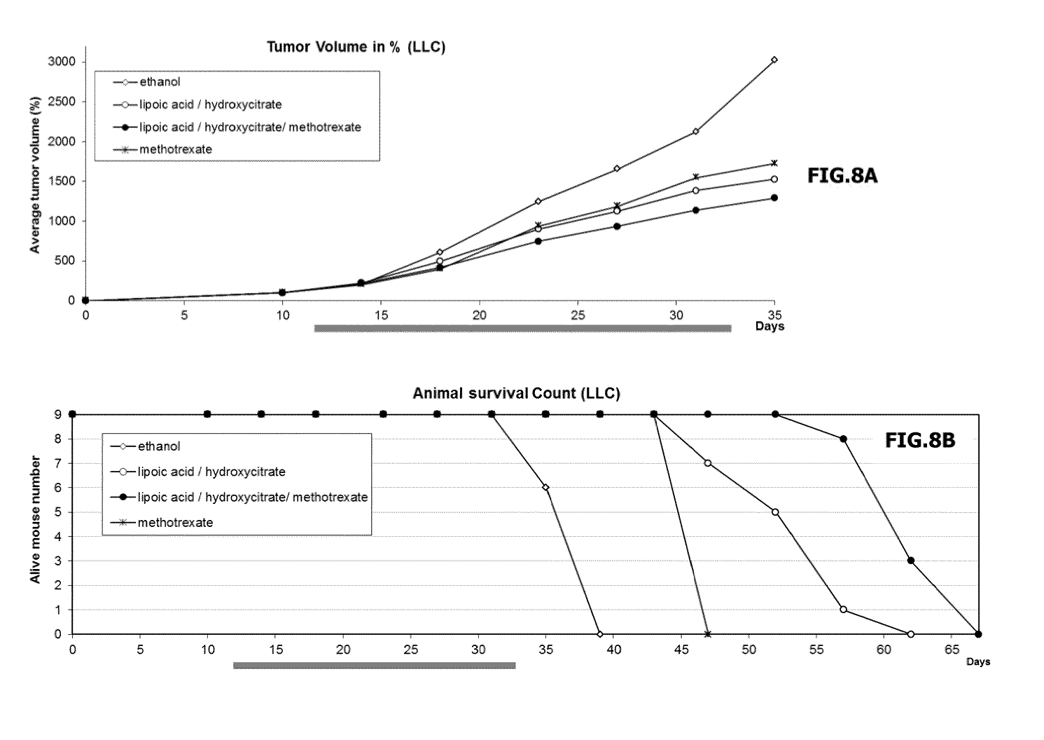

FIG. 8A: graphical representation representing the evolution of the average LLC tumor volume during a pharmaceutical treatment of the invention;

FIG. 8B: graphical representation representing the survival rate of the tested mice implanted with LLC type tumors during a pharmaceutical treatment of the invention.

### EXPERIMENTS CARRIED OUT

The notable antitumor activity of the therapeutic combination according to the invention was demonstrated by means of the experiments described hereinafter.

1—Tests on Human Tumor Cell Lines

1.1 Cell Lines Used

The human tumor cell lines and the culture media were obtained from the ATCC (American Type Culture Collection, Manassas, Va., United States).

The HT-29 tumor cell line was isolated in 1964 from a primary colonic adenocarcinoma in a 44-year-old woman (Fogh J. et al. 1977 J. Nat. Cancer. Inst. 59: 221-6).

The T-24 tumor cell line is a transitional bladder carcinoma isolated from an 81-year-old woman (O'Toole C M. et al. 1983 Nature 301: 429-30).

1.2—Culture Conditions

The tumor cell lines were cultured in monolayer at 37° C. in a humidified atmosphere (5% CO₂, 95% air). The culture medium which was used is DMEM Glutamax I (Invitrogen) for the two lines, supplemented with 10% of fetal calf serum (Eurobio) and 1/10 000 IU of penicillin and streptomycin. For carrying out the experiments, the human tumor cell lines were detached from the culture flask by treatment for 10 minutes with a solution of trypsin in Hanks medium without calcium or magnesium. The cells were counted in a hemocytometer and their viability was determined using the trypan blue exclusion test.

The tumor cell lines were amplified and then seeded into 96-well microplates (MTT) or 6-well plates (cell counts) at a concentration which allowed the cells to be in the proliferation phase for the 5 days of the culture. They were incubated for 48 hours before the beginning of the treatments in the microplates containing culture medium without the test substances or the reference substances.

During phase I (test with a single active ingredient), the tumor cell lines were incubated for 5 days at 37° C. under 5% CO₂ with culture medium containing one of the test substances. Each experimental condition was reproduced six times. Generally, the flasks of culture medium supplemented with serum were first of all prepared with the highest concentration of each test molecule. Each other concentration to be tested was obtained by successive dilutions in the serum-supplemented culture medium. This step was adjusted according to the particular instability or sensitivity of each drug.

In phase II of the study (test with the combination of the two active ingredients), the same conditions for preparing the media and for culturing the cells were adhered to, and the active ingredients were combined and added simultaneously to the culture medium.

The tumor cell lines were incubated with 100 μl (96-well plates) or 2 ml (6-well plates) of serum-supplemented culture medium containing the test substances or the reference substance. The culture medium of each culture well (supplemented with the test molecule(s)) was renewed every two days during the treatment.

1.3—Cell Proliferation and Viability Test

Starting from the first day of contact, and every two days after the beginning of the contacting of the culture media containing the test substances and the reference substances at the various concentrations tested, the proliferation and the cell viability of the cells were evaluated. The cell viability was evaluated in two different ways:
directly, under an optical microscope by statistical counting (number of cells), and
indirectly, by colorimetric assay based on the degradation of tetrazolium salts by mitochondrial dehydrogenases (MIT test). The optical densities (OD) of each well were then read with a microplate reader set at the appropriate wavelength.

1.4—Presentation of the Results

For each active ingredient, each concentration tested and each combination, three cell viability values were measured and corrected, in particular by means of the calibration ranges for the optical reading instrument.

The mean of the three cell viability measurement values was then divided by the mean of the measurements of viability carried out in parallel in a control culture free of active ingredients.

Table 1 hereinafter recaps the values calculated from the viability measurements made under an optical microscope or by luminescent labeling (number of cells/MTT) for the HT-29 cell line treated with lipoic acid alone or with calcium hydroxycitrate alone.

These values have also been reported in graph form in FIGS. 1A and 1B.

TABLE 1
Ratio of live HT-29 cells after 72 hours

| Molecules | | Cell line: HT-29 | | |
|---|---|---|---|---|
| tested alone | | Counting carried out after 72 hours | | |
| Lipoic acid | Concentrations | 0.1 | 1 | 10 |
| (FIG. 1A) | (μmol · l⁻¹) | | | |
| | MTT | 0.807 | 0.55 | 0.452 |
| | Number of cells | 0.853 | 0.583 | 0.441 |
| Calcium | Concentrations | 10 | 100 | 500 |
| hydroxycitrate | (μmol · l⁻¹) | | | |
| (FIG. 1B) | MTT | 0.743 | 0.519 | 0.376 |
| | Number of cells | 0.777 | 0.557 | 0.374 |

Table 2 hereinafter recaps the values calculated from the viability measurements made under an optical microscope or by luminescent labeling (number of cells/MTT) for the T-24 cell line treated with lipoic acid alone or with calcium hydroxycitrate alone.

These values have been reported in graph form in FIGS. 2A and 2B.

In the graphs of FIGS. 1A, 1B, 2A and 2B, the Y-axis indicates the percentage of live cells in each condition relative to a negative control (dilution vehicle) counted with a naked eye (number of cells) or by means of labeling of the live cells (MTT) after 72 hours. The X-axis indicates the concentration, expressed in micromoles per liter, of the active ingredient used.

TABLE 2
Ratio of live T-24 cells after 72 hours

| Molecules | | Cell line: T-24 | | |
|---|---|---|---|---|
| tested alone | | Counting carried out after 72 hours | | |
| Lipoic acid | Concentrations | 0.1 | 1 | 10 |
| (FIG. 2A) | (μmol · l⁻¹) | | | |
| | MTT | 0.898 | 0.690 | 0.534 |
| | Number of cells | 0.940 | 0.772 | 0.601 |
| Calcium | Concentrations | 0.2 | 3 | 45 |
| hydroxycitrate | (μmol · l⁻¹) | | | |
| (FIG. 2B) | MTT | 0.725 | 0.488 | 0.359 |
| | Number of cells | 0.845 | 0.643 | 0.450 |

Tables 3 and 4 hereinafter recap the values obtained by the two methods for measuring cell viability, using the combination of the ingredients according to the invention at various concentrations.

These values have been reported in graph form in FIGS. 3A to 3D.

In these graphs, the Y-axis indicates the percentage of live cells in each condition relative to a negative control (dilution vehicle) counted with the naked eye (number of cells) or by means of labeling of the live cells (MTT) after 72 hours.

The viability measurements were carried out at three increasing concentrations of calcium hydroxycitrate, respectively represented by the symbols: ▪ (100 micromol per liter), ▴ (200 micromol per liter) and ♦ (300 micromol per liter).

The X-axis indicates the concentration, expressed in micromoles per liter of lipoic acid used.

TABLE 3
Results obtained with the lipoic acid + (Ca) hydroxycitrate combination

| (Ca) | | Lipoic acid + (Ca) hydroxycitrate | | |
|---|---|---|---|---|
| hydroxycitrate | | live HT-29 cells after 72 hours | | |
| concentration | (FIG. 3B) | Lipoic acid concentration | | |
| (μmol · l⁻¹) | (FIG. 3A) | 4 | 8 | 16 |
| 100 | MTT | 0.177 | 0 | 0 |
| | Number of cells | 0.147 | 0 | 0 |
| 200 | MTT | 0.154 | 0 | 0 |
| | Number of cells | 0.085 | 0 | 0 |
| 300 | MTT | 0 | 0 | 0 |
| | Number of cells | 0 | 0 | 0 |

TABLE 4
Results obtained with the lipoic add + (Ca) hydroxycitrate combination

| (Ca) | | Lipoic acid + (Ca) hydroxycitrate | | |
|---|---|---|---|---|
| hydroxycitrate | | live T-24 cells after 72 hours | | |
| concentration | (FIG. 3D) | Lipoic acid concentration | | |
| (μmol · l⁻¹) | (FIG. 3C) | 4 | 8 | 16 |
| 100 | MTT | 0.229 | 0.142 | 0 |
| | Number of cells | 0.393 | 0.220 | 0 |
| 200 | MTT | 0.163 | 0.132 | 0 |
| | Number of cells | 0.271 | 0.195 | 0 |
| 300 | MTT | 0 | 0 | 0 |
| | Number of cells | 0 | 0 | 0 |

1.5—Observations Regarding the Results:

1.5.1—Active Ingredients Used Alone

The concentrations of active ingredient which were tested were defined according to the toxicological data available for each active ingredient. They correspond to doses which, in the event of the active ingredients being administered to humans orally, would not be toxic.

It is observed that, as a general rule, these active ingredients do not by themselves make it possible to induce 100% cell mortality, even at the highest concentrations tested.

1.5.2—Active Ingredients in Combination

It is observed that the results obtained for the two cell lines HT-29 and T-24 are about the same and that the two methods for counting live cells (number of cells/MTT) have very close profiles.

The effect of the combination of lipoic acid and hydroxycitrate on the cell viability of the HT-29 and T-24 lines is illustrated by the graphs of FIGS. 3A to 3D.

Taken separately, a concentration of 4 μmol.l⁻¹ of lipoic acid and of 100 μmol.l⁻¹ of calcium hydroxycitrate each make it possible, at best, to induce 50% cell mortality (FIGS. 1A and 1B, 2A and 2B).

On the other hand, when the lipoic acid and the hydroxycitrate are used in combined form, the cell mortality exceeds 80%. These results therefore show a synergistic effect of the combination of the two active ingredients.

A 100% cell mortality is achieved as soon as the lipoic acid concentration is increased to 8 μmol.l⁻¹ for a hydroxycitrate concentration of 200 μmol.l⁻¹.

2—Antitumor Activity Tests on C3H Mice

2.1—Murine Model

The compositions described hereinafter were tested against MBT-2 murine bladder tumors implanted in syngeneic C3H mice. These mice develop a tumor of 7 to 10 mm in diameter in approximately 20 days. The pharmaceutical combinations according to the invention and also the control compositions were administered intraperitoneally, for 21 days, starting from the 19th day after tumor inoculation. The change in tumor development was monitored by measuring the size of the tumors and monitoring the survival of the animals during the experiment.

The mice were randomized in groups of 18 individuals, the tumor of which is palpable (size of approximately 10 mm, after approximately 19 days of tumor development), according to the size of the tumor and the weight of the animals. For each animal, the maximum diameter of each tumor was measured with a Vernier caliper in order to determine the tumor volume.

The mice used in this study were treated in accordance with the ethical regulations in force.

2.2—Culture and Inoculation of Tumors

The MBT-2 tumor cell line is a transitional bladder carcinoma induced by FANFT (N-[4-(5-nitro-2-furyl)-2-thiazolyl]formamide) in a mouse of the C3H/HeN line (Soloway M S. et al. 1973 Surg. Forum. 24: 542-4).

The MBT-2 line was cultured in monolayer at 37° C. in a humidified atmosphere (5% CO₂, 95% air). The culture medium which was used is DMEM Glutamax I (Invitrogen) supplemented with 10% of fetal calf serum (Eurobio) and 1/10 000 IU of penicillin and streptomycin.

The cells were detached from the culture flask by means of a treatment for 10 minutes with a trypsin/EDTA solution and then counted.

The MBT-2 cell line was placed in culture at a density of 3×10³ cells/16 mm well of a 24-well plate, and then cultured in monolayer at 37° C. The cells were detached from the culture flask by means of a treatment for 10 minutes with a trypsin/EDTA solution and then counted.

The MBT-2 cells were dissociated in a cell suspension, and an injection was carried out via a 25-gauge diameter needle in the flank of a 6-week-old male C3H mouse: 10⁶ cells (120 μl). The treatment began nineteen days after the implantation.

2.3—Combinations Tested

The pharmaceutical combination according to the invention was tested in vivo:

The following products were used to prepare this combination:
alpha-lipoic acid (T1395 Sigma-Aldrich)
calcium hydroxycitrate (55128 Sigma-Aldrich)

Control mouse batches were treated using compositions comprising:
a pyridine analog used as a medicament in cancer treatment: 5-fluorouracil (5-FU) (Sigma F6627),
the isotonic saline solution (9 g/l) used for the intraperitoneal injection without active ingredient,
the vehicle for dissolving the active ingredients (0.05% ethanol) (neutral control).

The concentrations tested and the experimental conditions used are recapped in the following tables 5 and 6.

TABLE 5
Number of intraperitoneal injections per day and amounts
of molecules injected according to the conditions

| | Number of | |
|---|---|---|
| | injections per | Amount per |
| Active ingredients | day | injection |
| Alpha-lipoic acid | 2 | 10 mg/kg |
| Calcium hydroxycitrate | 2 | 250 mg/kg |

TABLE 6
Groups of animals tested and Corresponding experimental conditions

| | | | Duration |
|---|---|---|---|
| | | Number | of |
| Group | Treatment | of animals | treatment |
| 1 | Naive mice | 18 | — |
| 2 | Implantation medium alone | 18 | — |
| 3 | Saline solution | 18 | 21 days |
| 4 | Saline solution + 5-FU | 18 | 4 days |
| 5 | Saline solution + 0.05% ethanol | 18 | 21 days |
| 6 | Lipoic acid and calcium hydroxycitrate | 18 | 21 days |

After randomization of the animals in various groups, the treatments with the various active ingredients were carried out every day intraperitoneally (IP). Since the active ingredients have a short half-life (less than 12 hours), they were adminitered twice a day (morning and evening) (cf. table 5).

The treatment with 5-FU (positive control) was administered intraperitoneally with a dose of 10 mg/kg once a day for 4 days. The mice treated with 5-FU were monitored in the same way as the other mice.

2.4—Monitoring and Results

Every five days, the evolution of the tumor was monitored by various measurements: weight of the animals, tumor size (caliper rule), mortality.

A daily monitoring of the animals was carried out once a day and made it possible to determine precisely the day on which the animals died and to autopsy them rapidly. This monitoring also made it possible to isolate or euthanize the weak or moribund animals according to the recommendations of the EEC, of the ASAB, of the Canadian Council on Animal Care and of the UKCCCR.

The results of measurement of the average tumor volume in each of the groups of treated mice, as a function of time, are reported in the graph of FIG. 4A.

In this graph, the Y-axis indicates the increase in percentage of average volume (relative to the volume measured on the first day of treatment) of the tumors measured in the mice.

The X-axis indicates the number of days for which the mice were monitored.

The results showing the survival of the mice treated in this study are reported in the graph of FIG. 4B.

In this graph, the Y-axis indicates the number of mice alive.

The X-axis indicates the number of days for which the mice were monitored.

Moreover, in FIGS. 4A and 4B:
the hatched part between days 19 and 40 represents the period during which the treatment was administered;
the following symbols were used:
   ∘: lipoic acid+calcium hydroxycitrate;
   ⋄: saline solution control;
   *: 5-FU;
   Δ: ethanol control.

2.5—Interpretation of the Results

As shown by the graph of FIG. 4A, the pharmaceutical combination according to the invention makes it possible to limit the growth of the tumors, the volume of which stabilizes, over a period of at least 100 days, at values of 100% of the volume reached by the tumors at the beginning of the treatment.

This result should be compared with the volume reached by the tumors in the control mice, the increase of which is of the order of 500%.

It is interesting to note that the treatment according to the invention made it possible, entirely surprisingly and unexpectedly, to obtain a significantly greater tumor stabilization effect than that obtained using 5-FU, which is an anticancer medicament.

As shown by the graph of FIG. 4B, the pharmaceutical combination according to the invention made it possible to significantly increase the survival time of the treated mice.

Thus, in the groups corresponding to the saline-solution and ethanol controls, approximately 50% of the mice are still alive after 27 days of implantation.

In the group treated with 5-FU, 50% of the mice are still alive after 39 days of implantation, which corresponds to an increase in survival of 12 days.

In the group of mice treated with the pharmaceutical combination according to the invention, 50% of the mice are still alive after 74 days, which corresponds to an increase in survival of 35 days compared with 5-FU (survival time multiplied by 1.9) and of 47 days compared with the control mice (survival time multiplied by 2.7).

Thus, it is interesting to note that the treatment according to the invention made it possible, entirely surprisingly and unexpectedly, to increase the survival rate of the mice in a significantly large manner compared with the treatment using 5-FU.

3—Results Obtained with Other Additional Active Ingredients

Complementary tests were carried out in order to evaluate the efficacy of a pharmaceutical combination incorporating lipoic acid or one of the pharmaceutically acceptable salts thereof, hydroxycitric acid or one of the pharmaceutically acceptable salts thereof, and at least one additional active ingredient, in particular an active ingredient having an antitumor activity.

These additional experiments demonstrated the fact that the efficacy of a combination of lipoic acid (or one of the pharmaceutically acceptable salts thereof) and of hydroxycitric acid (or one of the pharmaceutically acceptable salts thereof) can be further improved when these two active ingredients are combined with an additional active ingredient, in particular chosen from the group consisting of cisplatin, capsaicin, choline, miltefosine and vitamin B12.

These tests were carried out on the MBT-2 (bladder carcinoma) or LL/2 (lung carcinoma) models implanted in the back of syngeneic mice (9 mice/group).

When the tumor volume reached approximately 100 mm³, the mice were treated intraperitoneally for 3 weeks according to the following doses:
hydroxycitric acid (hereinafter HCA) 250 mg/kg twice a day,
lipoic acid (hereinafter ALA) 10 mg/kg twice a day,
cisplatin 1 mg/kg every other day,
capsaicin 5 mg/kg or 750 μg/kg once a day,
miltefosine 20 mg/kg/day,
vitamin B12 5 μg/kg/day.

Tumor development was monitored by regularly measuring the dimensions of the tumor, and the inhibition of tumor growth was calculated by the % T/C ratio (ratio between the average tumor volume of the treated group compared with the control group at a given time).

At the end of these tests, it was observed that:
The use of a combination of ALA, HCA and cisplatin makes it possible to reduce the tumor development by 40% compared with the ALA/HCA combination alone (MBT-2 model).
The use of a combination of ALA, HCA and capsaicin (5 mg/kg/day) makes it possible to reduce the tumor development by 66% compared with the ALA/HCA combination alone (LL/2 model).
The use of a combination of ALA, HCA, capsaicin (750 μg/kg/day) and cisplatin makes it possible to reduce the tumor development by 32% compared with the ALA/HCA combination alone (LL/2 model).
The use of a combination of ALA, HCA, capsaicin, miltefosine, choline and vitamin B12 makes it possible to reduce the tumor development by 21% compared with the ALA/HCA combination alone (LL/2 model).

3—Further Antitumor Activity Tests on C3H and C57BL/6J Mice (Corresponding to FIG. 5A through to 8B)

3.1—Murine Model

The compositions described hereinafter were tested against two syngeneic tumor models:
A bladder carcinoma MBT-2 implanted in syngeneic C3H mice,
A pulmonary carcinoma LL/2 (or LLC1) called <<Lewis carcinoma>> implanted in syngeneic C57BL/6J mice,

Every 4 to 5 days, the survival and the tumor volume were observed. For each animal, the two greater diameters and the greater height of each tumors were measured using a Vernier caliper so that to determine the tumor volume (according to the formula: length×width×height×pi/6).

Mice used in this study were treated in accordance with ethical regulations in force. A daily monitoring a the mice was carried out so that careful determination of the date and time of death could be achieved and the autopsy could be performed quickly. This careful monitoring also allowed for the isolation or the euthanesia of the weak or moribond animals according to CEE, ASAB, Canadian Council on Animal Care and I′UKCCCR recomendations.

3.2—Culture and Inoculation of Tumors

3.2.1—Cell Lines

The MBT-2 tumor cell line is a transitional bladder carcinoma induced by FANFT (N-[4-(5-nitro-2-furyl)-2-thiazolyl]formamide) in a mouse of the C3H/HeN line (Soloway M S. et al. 1973 Surg. Forum. 24: 542-4) obtained from a state-owned laboratory.

The LL/2 tumor cell line (ATCC n^{o} CRL-1642) is a murin pulmonary carcinoma (Lewis carcinoma) obtained from the ATCC (LGC Promochem).

3.2.2—Culture and Implantation

The four cell lines were cultured in monolayer at 37° C. in a humidified atmosphere (5% CO₂, 95% air). The culture medium which was used is DMEM Glutamax I (Invitrogen) supplemented with 10% of fetal calf serum (Eurobio) and 1/10 000 IU of penicillin and streptomycin.

The cells were detached from the culture flask by means of a treatment for 10 minutes with a trypsin/EDTA solution and then counted.

The cells were then dissociated in a cell suspension (10⁶ cells were injected into 120 μL), and an injection was carried out via a 25-gauge diameter needle in the flank of the mouse.

When the tumor volumes reached 95-100 mm³, the mice were randomised according to the size of the tumor and of the weight of each mouse.

For each each animal, the two greater diameters and the height of each tumor were measured using a Vernier caliper to determine the tumor volume (according to the formula <<length×width×height×pi/6>> or <<length×width²/2>>).

3.3—Tested Combinations

The pharmaceutiol combination of the invention was tested in vivo:

The following products were used to prepare this combination:
alpha-lipoic acid (T1395 Sigma-Aldrich)
Calcium/potassium hydroxycitrate (Garcinia cambodia extract, 60% HCA, Indo World Trade company)
Capsaïcine (Sigma-Aldrich 12084)
A platinum-containing organometallic complex used as an active agent in the treatment of cancer: cisplatine (CIS) (Sigma 479306),
Methotrexate (Sigma-Aldrich M9929)

Groups of control mice were treated using compositions comprising:
An isotonic salt solution (9 g/L) used for the intraperitoneal injection without any active agents,
A dissolution carrier for the actives agents (ethanol 0.5%) (control).

The tested concentrations and the experimental conditions used in the present study are summarised in the following tables 7 et 8.

TABLE 7
Number of intraperitoneal injections per day and amounts of
molecules injected according to the conditions.

| | Abbre- | | Amount per | Experimental |
|---|---|---|---|---|
| Active agents | viation | Injection rate | injection | model |
| alpha-lipoic | ALA | Every 12 hours | 10 mg/kg | MBT-2, |
| acid | | | | LLC |
| Ca/K | HCA | Every 12 hours | 250 mg/kg | MBT-2, |
| Hydroxycitrate | | | | LLC |
| Cisplatine | CIS | Every other | 1 mg/kg | MBT-2, |
| | | day | | LLC |
| Methotrexate | MTX | Every 24 hours | 1 mg/kg | LLC |
| Capsaïcine | CAP | Every 24 hours | 0.75 mg/kg | LLC |

TABLE 8
Groups of animals tested and corresponding experimental conditions

| Groups | treatment | Experimental model |
|---|---|---|
| 1 | Salt solution + ethanol 0.5% | MBT-2, LLC |
| 2 | CIS | MBT-2, LLC |
| 3 | MTX | LLC |
| 4 | ALA × HCA | MBT-2, LLC |
| 5 | ALA × HCA × MTX | LLC |
| 6 | ALA × HCA × CIS | MBT-2, LLC |
| 7 | ALA × HCA × CIS × CAP | LLC |

3.4—Results

For the four tumor models herein described, the results corresponding to the measured average tumor volume in each of the treated mouse group, plotted against the treatment duration, are shown in FIGS. 5A, 6A, 7A et 8A.

As shown in the graphs, the Y-axis indicates the increase of average tumor volume in percent (relative to the tumor volume as measured on the first day of treatment) as measured in the tested mice. The X-axis indicates the number of days during which the mice have survived starting from the tumor implantation. Further, the greyed area on each graph represent the treatment duration i.e. the period of time during which the treatment was administered.

The results showing the survival rate of the traited mice in the present study are summarised in FIGS. 5B, 6B, 7B and 8B.

In these graphs, the Y-axis indicates the number of mice which have survived. The X-axis indicates the number of days during which the mice have managed to survive from the tumor implantation. Further, the greyed area on each graph represent the treatment duration i.e. the period of time during which the treatment was administered.

3.5—Results Discussion

3.5.1. Efficiency of the ALA/HCA Treatment when Used with Cisplatine

In the MBT-2 model (FIG. 5A), the treatment using the ALA/HCA/CIS combination resulted in a 70% decrease of the tumor volume relative to the control ethanol (60 days).

This tumor growth reduction observed when using the ALA/HCA/CIS treatment is associated with a significant increase of the mice life-span (FIG. 5B) i.e. with a survival time increase of up to about 31 days between control animals (100% of survival during 55 days) and animals traited with the ALA/HCA/CIS treatment (100% of survival during 86 days).

In the LLC model (FIG. 6A), the ALA/HCA/CIS treatment enabled the average tumor volume to be decreased by more than 43% relative to the average tumor volume measured in mice groups using the control ethanol (55 days).

This tumor growth reduction observed when using the ALA/HCA/CIS treatment is associated with a significant increase of the mice life-span (FIG. 6B) i.e. with a survival,time increase of up to about 10 days between control animals (100% of survival during 50 days) and animals treated with the ALA/HCA/CIS treatment (100% of survival during 60 days).

3.5.2. Efficiency of the ALA/HCA/CAP Treatment when Used with Cisplatine

In the LLC model (FIG. 7A), the ALA/HCA/CIS/CAP treatment enabled the average tumor volume to be decreased by more than 59% relative to the average tumor volume measured in mice groups using the control ethanol (57 days).

This tumor growth reduction observed when using the ALA/HCA/CIS/CAP treatment is associated with a significant increase of the mice life-span (FIG. 7B) i.e. with a survival time increase of up to about 35 days between control animals (89% of survival during 65 days) and animals treated with the ALA/HCA/CAP/CIS treatment (89% of survival during 100 days).

3.5.3. Efficiency of the ALA/HCA Treatment when Used with Methotrexate

In the LLC model (FIG. 8A), the ALA/HCA/MTX treatment enabled the average tumor volume to be decreased by more than 56% relative to the average tumor volume measured in mice groups using the control ethanol (35 days).

This tumor growth reduction observed when using the ALA/HCA/MTX treatment is associated with a significant increase of the mice life-span (FIG. 8B) i.e. with a survival time increase of up to about 10 days between control animals (100% of survival during 30 days) and animals treated with the ALA/HCA/MTX treatment (100% of survival during 52 days).

4—Antitumor Activity Tests on Human Patients

The ALA/HCA combination of the invention was administered to a number of cancer patients alone or with one or more other active agents (such as the ones usually used in chemotherapy treatments) and we describe here the data that were obtained.

### 4.1. Patient 1: Metastatic Pancreatic Cancer

This patient was a 80 year-old female with pancreatic cancer showing liver metastasis (TNM staging: pt3 pN1 (12/28) M1; G3). She was treated with a treatment using, amongst others, the combination of the present invention (see table 9) during 8 months and showed a meaningful improvement of the survival time relative to the estimated survival time without treatment.

An amelioration of all parameters was observed during several months, such as an increase of body weight, a decrease of CA19-9 tumor marker and a regression of liver tumor observed by CT-scan.

After 5 months, the patient decided to modify her treatment regimen by taking herself off the treatment of some of the administered drugs. Three months later, the tumor reappeared.

This lead to the administration of a new chemotherapy regimen, FOLFOX, associated with ALA/HCA and others (see table 10). The disease stabilized again during six more months until it progressed again. Overall, the patient of the present study survived 18 months after diagnosis of its pancreas cancer while her estimated survival was 3-6 months according to diagnosis based on clinical observations and CA19-9 tumor marker values.

TABLE 9
Drugs and dosage administered to patient 1 -
This treatment was administered during an 8-month period.

| Drugs | Dosage/schedule |
|---|---|
| Gemcitabine | 1200 mg every 28 days |
| Garcinia Cambogia (60% | 1200 mg per os every day |
| HCA) | |
| α lipoic acid (tiobec ®) | 1200 mg per os every day |
| Celecoxib (Celebrex ®) | 200 mg per os per day |
| Retinoic acid | 50 mg per os every other day |
| Melatonin | 20 mg per os per day at 9 pm |
| Prosure ® (Abbott) | 2 vials per os per day |

TABLE 10
Drugs and dosage administered after tumor reappearance in patient
1 - This treatment was administered for the last 6 months of treatment.

| Drugs | Dosage/schedule |
|---|---|
| FOLFOX (Folinic acid, 5- | 2-week cycle: day 1, concomitant leucovorin |
| fluorouracil, oxaliplatin) | 300 mg/m² and oxaliplatin |
| | 120 mg/m² IV infusions followed-up |
| | by 5-FU 600 mg/mg²; day 2 of leucovorin |
| | followed-up by 5-FU, same doses |
| Garcinia Cambogia (60% | 1200 mg per os every day |
| HCA) | |
| α lipoic acid (tiobec ™) | 1200 mg per os every day |
| Celecoxib (Celebrex ™) | 200 mg per os per day |
| Retinoic acid | 50 mg per os every other day |
| Melatonin | 20 mg per os per day at 9 pm |
| Naltrexone | 1 mg per os per day at 9 pm |
| Prosure ™ (Abbott) | 2 vials per os per day |

### 4.2. Patient 2: Metastatic Breast Cancer

This patient was a 53 year-old female with breast cancer with metastases (TNM staging: pT2 N2 M1). After surgery and several sessions of radiotherapy and chemotherapy the disease reappeared showing metastases, notably in bones and liver.

The patient was then administered with a combination of several drugs, among them docetaxel, capecitabin and the ALA/HCA combination (see Table 11). After 9 months, the docetaxel/capecitabin combination was replaced by epirubicine, the other drugs being maintained. Thirteen months after the beginning of the ALA/HCA combination, the disease was still stable.

TABLE 11
Drugs and dosage prescribed to patient 2

| Drugs | Dosage/schedule |
|---|---|
| Docetaxel | 75 mg/mq every 28 days |
| Capecitabin | 1500 mg per os every day |
| Garcinia Cambogia (60% HCA) | 1200 mg per os every day |
| α lipoic acid (tiobec ®) | 1200 mg per os every day |
| Melatonin | 20 mg per os per day at 9 pm |
| Trans Retinoic acid | 50 mg per os every other day |
| Wobenzym ® | 2 cps per os per day at morning |
| Silibinum | 200 2 times a day |

### 4.3. Patient 3: Glioblastoma

This patient was a 39 year-old female with glioblastoma in the temporal frontal area (TNM staging: pT4 N0 M0). After surgery and several sessions of radiotherapy and chemotherapy the disease was still progressing. The patient was then admninstered with a combination of several drugs, among them thalidomide and the ALA/HCA combination (see Table 12). Nine months after the beginning of this therapy, the disease was still stable.

TABLE 12
Drugs and dosage prescribed to patient 3

| Drugs | Dosage/schedule |
|---|---|
| Thalidomide | 20 mg per os every day |
| Garcinia Cambogia (60% HCA) | 1200 mg per os every day |
| α lipoic acid (tiobec ®) | 1200 mg per os every day |
| Melatonin | 20 mg per os per day at 9 pm |
| Boswelia | 400 per os every day |

### 4.4. Patient 4: Metastatic Parotid Gland Cancer

This patient is a 55 year-old male with parotid gland cancer (TNM staging: pT3b N2). Subtotal excision revealed a poorly differentiated (Grade 3) pleomorphic carcinoma of the parotid. After surgery and several sessions of radiotherapy and chemotherapy the disease progressed with signs of metastases, notably in the brain.

The patient was then administered weekly with epirubicine and the ALA/HCA combination (lipoic acid, 600 mg three times a day, and hydroxycitrate from Garcinia Cambodgia, 1 gr three times a day). A partial remission was observed with a decrease at all tumor sites.

Three months later, epirubicine was replaced by vinorelbin and gemcitabine combined with the ALA/HCA combination with a very good partial remission.

Six months after of vinorelbin and gemcitabine combined with ALA/HCA treatment, it was stopped with an almost complete disappearance of the tumor (90%).

The patient then returned to a normal life and regained 10 kilograms.

Two months after treatment interruption, a local relapse in the parotid with a brain metastasis was observed. Chemotherapy (vinorelbin and gemcitabine) in combination with the combination of the present invention ALA/HCA was administered again.

A regression of the tumor mass and of the brain metastasis was observed which was treated by Gamma knife one month after chemotherapy restart.

Four months after the chemotherapy had resumed, the tumor was still being treated but was stable.

### 4.5. Patient 5: Metastatic Breast Cancer

This patient was a 50 year-old female with breast cancer with metastases to bones. After surgery and several sessions of radiotherapy and chemotherapy the disease recurred showing metastases.

After several years of treatment, the patient refused to be treated with any kind of usual chemotherapy treatment. So she was only administered with a ALA/HCA combination (lipoic acid, 400 mg a day, and hydroxycitrate from Garcinia Cambodgia, 1.2 gr a day). Thirteen months after the beginning of the ALA/HCA combination, the progression of the disease was observed to be significantly impaired.

The pharmaceutical combination of the present invention is therefore useful in the treatment of cancer when used alone or together with another anticancer treatment as exemplified using cancer cell lines, animal studies and human studies.

### Example of a Pharmaceutical Composition According to the Invention

A pharmaceutical composition according to the invention can be, for example, formulated in the form of a gelatin capsule containing the following ingredients:
50 mg of alpha-lipoic acid,
400 mg of hydroxycitric acid,
109 mg of a shell consisting of hydroxypropylmethylcellulose,
20 mg of anti-aggregating agents, combination of plant magnesium stearate and of silicon dioxide,
15 mg of binder (hydroxypropylcellulose).

Such a composition can be administered according to this dosage at a rate of 2 gelatin capsules, 3 times a day, a minimum of one hour before meals.

1. A method for the treatment of tumors comprising a step of administering to a patient in need thereof a pharmaceutical combination comprising, as active ingredients:
lipoic acid or one of the pharmaceutically acceptable salts thereof; and
hydroxycitric add or one of the pharmaceutically acceptable salts thereof;
said active ingredients being formulated together or separately, for a conjugated, simultaneous or separate administration.

2. The method as claimed in claim 1, wherein said hydroxycitric acid salt is an alkali metal or alkaline-earth metal salt.

3. The method as claimed in claim 1, wherein said hydroxycitric acid salt is a calcium salt.

4. The method as claimed in claim 1, wherein said active ingredients are provided:
together, in a galenical form suitable for oral administration; or
separately, independently of one another, each in a galenical form suitable for oral administration.

5. The method as claimed in claim 4, wherein said galenical form suitable for oral administration is selected from the group consisting of tablets, gelatin capsules, powders, granules, lyophilisates, oral solutes and syrups.

6. The method as claimed in claim 1, wherein said pharmaceutical combination is in a unitary form in which the said active ingredients, together, are in the form of tablets.

7. The method as claimed in claim 1, comprising:
lipoic acid or one of the pharmaceutically acceptable salts thereof in an amount of between 20 and 800 mg;
hydroxycitric acid or one of the pharmaceutically acceptable salts thereof in an amount of between 200 and 2000 mg.

8. The method as claimed in claim 1, wherein said pharmaceutical combination further comprises at least one additional active ingredient selected from the group consisting of cisplatin, capsaicin, choline, miltefosine, methotrexate and vitamin B12.

9. The method as claimed in claim 8, wherein said additional active ingredient is selected from the group consisting of cisplatin, capsaicin and methotrexate.

10. The method as claimed in claim 8, wherein said pharmaceutical combination comprises both cisplatin and capsaicin.

11. The method as claimed in claim 8, wherein said pharmaceutical combination comprises all of capsaicin, vitamin B12, choline and miltefosine.

12. The method as claimed in claim 2, wherein said hydroxycitric acid salt is a calcium salt.

13. The method as claimed in claim 2, wherein said active ingredients are provided:
together, in a galenical form suitable for oral administration; or
separately, independently of one another, each in a galenical form suitable for oral administration.

14. The method as claimed in claim 2, wherein said pharmaceutical combination is in a unitary form in which the said active ingredients, together, are in the form of tablets.

15. The method as claimed in claim 4, wherein said pharmaceutical combination is in a unitary form in which the said active ingredients, together, are in the form of tablets.

16. The method as daimed in claim 2, comprising:
lipoic acid or one of the pharmaceutically acceptable salts thereof in an amount of between 20 and 800 mg;
hydroxycitric acid or one of the pharmaceutically acceptable salts thereof in an amount of between 200 and 2000 mg.

17. The method as maimed in claim 4, comprising:
lipoic acid or one of the pharmaceutically acceptable salts thereof in an amount of between 20 and 800 mg;
hydroxycitric acid or one of the pharmaceutically acceptable salts thereof in an amount of between 200 and 2000 mg.

18. The method as claimed in claim 6, comprising:
lipoic acid or one of the pharmaceutically acceptable salts thereof in an amount of between 20 and 800 mg;
hydroxycitric acid or one of the pharmaceutically acceptable salts thereof in an amount of between 200 and 2000 mg.

19. The method as claimed in claim 2, wherein said pharmaceutical combination further comprises at least one additional active ingredient selected from the group consisting of capsaicin, choline, miltefosine, methotrexate and vitamin B12.

20. The method as claimed in claim 4, wherein said pharmaceutical combination further comprises at least one additional active ingredient selected from the group consisting of cisplatin, capsaicin, choline, miltefosine, methotrexate and vitamin B12.

21. The method as claimed in claim 6, wherein said pharmaceutical combination further comprises at least one additional active ingredient selected from the group consisting of cisplatin, capsaicin, choline, miltefosine, methotrexate and vitamin B12.

22. The method as claimed in claim 7, wherein said pharmaceutical combination further comprises at least one additional active ingredient selected from the group consisting of cisplatin, capsaicin, choline, miltefosine, methotrexate and vitamin B12.

23. The method as claimed in claim 1, wherein the pharmaceutical combination is used in a therapeutic treatment as a supplement to other anticancer treatments.

24. The method as claimed in claim 1, wherein the pharmaceutical combination is used in a therapeutic treatment as a supplement to other anticancer treatments.

25. The method as claimed in claim 2, wherein the pharmaceutical combination is used in a therapeutic treatment as a supplement to other anticancer treatments.

26. The method as claimed in claim 4, wherein the pharmaceutical combination is used in a therapeutic treatment as a supplement to other anticancer treatments.

27. The method as claimed in claim 6, wherein the pharmaceutical combination is used in a therapeutic treatment as a supplement to other anticancer treatments.

28. The method as claimed in claim 7, wherein the pharmaceutical combination is used in a therapeutic treatment as a supplement to other anticancer treatments.
